SEQUENCE LISTING (13 sequences)

SEQ ID NO: 1 - DNA, 21 bp, Artificial Sequence
  Primer
aaaaagccgcccgacctacct

SEQ ID NO: 2 - DNA, 22 bp, Artificial Sequence
  Primer
gctacaacgccgaaatgacctt

SEQ ID NO: 3 - DNA, 17 bp, Artificial Sequence
  Primer
gccgtcgcccagcactc

SEQ ID NO: 4 - DNA, 641 bp, Guignardia Citricarpa
tttccgtaggtgaacctgcggaaggatcattactgaaatacgtaatcctgaaaggtgatggaagggaggccttaaaaaagccgcccgacctaccttcacacccttgtgtatctaccatgttgctttggcgggccgacccggttttgacccgggcggtcggcgcccccagcctagtctctaggccaggacgcctggctaagtgcccgccagtatacaaaactccagcgattattctgtgtagtcctgagaattcatttaatgaaataaaactttcaacaacggatctcttggttctggcatcgatgaagaacgcagcgaaatgcgataagtaatgtgaattgcagaattcagtgaatcatcgaatctttgaacgcacattgcgccccctggcattccggggggcatgcctgttcgagcgtcatttcaaccctcaagctctgcttggtattgggcgacgtccgctgccggacgcgcctggaagacctcggcgacggcgtctcagcctcgagcgtagtagtaaaatatctcgctttggaggaggggggcgctggccgccggacaatcgaccttcggtcactatttttccaaggttgacctcggatcaggtagggatacccgctgaacttaagcatatcaataag

SEQ ID NO: 5 - DNA, 618 bp, Guignardia Citricarpa
ttccgtaggtgaacctgcggaaggatcattactgaaatgtaataacttctattgaaaggttccagagtaggcgctacaacgccgaaatgaccttctcacccttgtgtactcactatgttgctttggcgggtcgacctggttccgacccaggcggccggcgcccccagccttaactggccaggacgcccggctaagtgcccgccagtatacaaaactcaagaattcattttgtgaagtcctgatatatcatttaattgattaaaactttcaacaacggatctcttggttctggcatcgatgaagaacgcagcgaaatgcgataagtaatgtgaattgcagaattcagtgaatcatcgaatctttgaacgcacattgcgccctctggtattccggagggcatgcctgttcgagcgtcatttcaaccctcaagctctgcttggtattgggcaacgtccgctgccggacgtgccttgaagacctcggcgacggcgtcctagcctcgagcgtagtagtaaaatatctcgctttggagtgctgggcgacggccgccggacaatcgaccttcggtctatttttccaaggttgacctcggatcaggtagggatacccgctgaactt

SEQ ID NO: 6 - DNA, 20 bp, Artificial Sequence
  Primer
gctgcgttcttcatcgatgc

SEQ ID NO: 7 - DNA, 20 bp, Artificial Sequence
  Primer
gcatcgatgaagaacgcagc

SEQ ID NO: 8 - DNA, 21 bp, Artificial Sequence
  Primer
taaaaaagccgcccgacctac

SEQ ID NO: 9 - DNA, 20 bp, Artificial Sequence
  Primer
gctacaacgccgaaatgacc

SEQ ID NO: 10 - DNA, 20 bp, Artificial Sequence
  Primer
atgccagaaccaagagatcc

SEQ ID NO: 11 - DNA, 22 bp, Artificial Sequence
  Primer
tgcaattcacattacttatcgc

SEQ ID NO: 12 - DNA, 20 bp, Artificial Sequence
  Primer
tcctccgcttattgatatgc

SEQ ID NO: 13 - DNA, 22 bp, Artificial Sequence
  Primer
ggaagtaaaagtcgtaacaagg

(19) United States
(12) Patent Application Publication
(10) Pub. No.: US 2002/0172945 A1
(43) Pub. Date: Nov. 21, 2002

(54) Materials and methods for detection of pathogenic guignardia citricarpa

(76) Inventor: George C. Carroll, Eugene, OR (US)

Correspondence Address: DANN DORFMAN HERRELL & SKILLMAN, SUITE 720, 1601 MARKET STREET, PHILADELPHIA, PA, 19103-2307, US

(21) Appl. No.: 09/766,173

(22) Filed: Jan. 22, 2001

Related U.S. Application Data
(60) Provisional application No. 60/177,013, filed on Jan. 19, 2000.

(51) Int. Cl. C12Q 1/68; C07H 21/04; C12P 19/34

(52) U.S. Cl. 435/6; 435/91.2; 536/23.7

(57) ABSTRACT

Materials and methods are provided which rapidly and specifically differentiate between pathogenic and non-pathogenic fungi associated with Citrus Black Spot Disease.

### CROSS-REFERENCE TO RELATED APPLICATION

This application claims priority under 35 U.S.C. §119(e) to US Provisional Application 60,177,013 filed Jan. 19, 2000, the entire disclosure of which is incorporated by reference herein.

### FIELD OF THE INVENTION

This invention relates to the fields of molecular biology and the detection of detrimental fungal species. More specifically, the invention provides materials and methods which facilitate the differentiation of pathogenic Guignardia citricarpa species from non-pathogenic Guignardia species.

### BACKGROUND OF THE INVENTION

Black Spot of Citrus is a cosmetic disease on fruit of most of the commercial Citrus cultivars, including oranges, lemons, limes, and grapefruit. It begins with tiny black pustules on the surface of the developing fruit and, at room temperature, progresses to form highly developed, even all-encompassing black surface lesions. Although the disease originated in Africa, it has now spread to many areas of the world where Citrus are grown, including Brazil, Argentina, and China. The disease has not been reported in the United States and continued exclusion of the causative pathogen is a major concern of the USDA Plant Quarantine Service. The European Community has also adopted stringent measures to exclude spotted fruit from its borders.

The causative agent of Citrus Blackspot, Guignardia citricarpa (asexual state=Phyllosticta citricarpa) is a representative of a large and ubiquitous group of leaf-spotting Ascomycetes. Over 600 species of Phyllosticta have been reported in the United States, although many of these species descriptions are inadequate, and the actual number probably lies between 100-200. Phyllosticta spp. commonly form latent infections in many non-host plants, and are among the most commonly isolated asymptomatic fungal endophytes in leaves. For many years considerable confusion surrounded work on the taxonomic identity of Guignardia citricarpa and its pathogenicity on Citrus fruit. This confusion was finally resolved when it was discovered that there are actually two species of Guignardia on Citrus, and that one fruit, e.g., a single orange, may be infected simultaneously with both species. One species, the true G. citricarpa, is pathogenic, causes black spots on fruit, and is believed to be confined to the genus Citrus. The second species is non-pathogenic, forms insignificant lesions on fruit, and is widely dispersed on a huge number of unrelated host plants. The two fungal species are morphologically almost indistinguishable.

It would be highly advantageous to the Citrus fruit industry if means were available to differentiate between these two species of Guignardia.

### SUMMARY OF THE INVENTION

Materials and methods are provided which facilitate the differentiation between pathogenic and non-pathogenic species of Guignardia.

In a preferred embodiment of the invention, oligonucleotides are provided which specifically and selectively hybridize with pathogenic vs. non-pathogenic species of Guignardia. In one embodiment, the oligonucleotides are used for priming a DNA amplification of a target DNA sequence associated with pathogenic Guignardia is provided. Suitable oligonucleotide forward primers may be selected from the group consisting of those having the sequences AAAAAGCCGCCCGACCTACCT (SEQ ID NO: 1) and TAAAAAAGCCGCCCGACCTAC (SEQ ID NO: 8). Also provided is an oligonucleotide primer for forward priming a DNA amplification of a target DNA sequence associated with non-pathogenic Guignardia citricarpa. Suitable oligonucleotides may be selected from the group consisting of GCTACAACGCCGAAATGACCTT (SEQ ID NO: 2), GCCGTCGCCCAGCACTC (SEQ ID NO: 3), and GCTACAACGCCGAAATGACC (SEQ ID NO: 9). The following reverse primers are suitable for use in the amplification reactions disclosed above; SEQ ID NO: 6, SEQ ID NO: 10, and SEQ ID NO: 11.

In yet another embodiment of the invention, a method is disclosed for differentiating between pathogenic and non-pathogenic species of Guignardia in Citrus isolates. The method comprises i) obtaining a DNA sample from a Citrus fruit suspected of being infected with Guignardia; ii) contacting the fungal DNA with detectably labeled nucleic acid probes which bind pathogenic and non-pathogenic species of Guignardia selectively. Binding of a target fungal sequence being detectable by measuring the amount of detectably labeled probe bound. An alternative method comprises, i) obtaining a DNA sample from a Citrus fruit suspected of being infected with Guignardia and immobilizing the DNA on a solid support; iii) contacting the immobilized sample DNA with a detectably labeled oligonucleotide probe having a sequence selected from the group consisting of SEQ ID NO: 1, SEQ ID NO: 2, SEQ ID NO: 3, SEQ ID NO: 8, and SEQ ID NO: 9; and iv) assessing the solid support for hybridization of the probe to the immobilized DNA. Samples demonstrating hybridization with SEQ ID NOS: 1 or 8 are associated with pathogenic Guignardia infection. Samples demonstrating hybridization with SEQ ID NOS: 2, 3, or 9 are associated with the non-pathogenic Guignardia species.

Also within the scope of the present invention, are materials and methods for selectively amplifying nucleic acid sequences which correspond to pathogenic Guignardia citricarpa. For amplification of DNA associated with pathogenicity, the fungal isolate is cultured for a suitable time period. The hyphae are harvested and lysed, and the DNA is isolated from the lysate. Alternatively, DNA may be directly isolated from the black spot lesion. The isolated DNA is then incubated with a primer pair which comprises forward primers SEQ ID NO: 1 or SEQ ID NO: 8 and a reverse primer selected from the group consisting of SEQ ID NO: 6 and TGCAATTCACATTACTTATCGC (SEQ ID NO: 11) under conditions suitable for PCR amplification. Preferably the primer pairs are SEQ ID NOS: 8 and 11. Also provided are materials and methods for selectively amplifying nucleic acid sequences which correspond to the non-pathogenic Guignardia species. For amplification of DNA associated with such non-pathogenic isolates, a suitable forward primer comprises SEQ ID NO: 2 or SEQ ID NO: 9 and a reverse primer selected from the group consisting of SEQ ID NO: 6, ATGCCAGAACCAAGAGATCC (SEQ ID NO: 10), and SEQ ID NO: 11. Preferably, the primer pairs are SEQ ID NOS: 9 and 10.

In a particularly preferred embodiment, kits for practicing the methods of the invention are provided. Components of such a kit include a pathogen-specific oligonucleotide probe, optionally immobilized on solid matrix, including means for amplifying the target fungal nucleic acid in a test sample, the nucleic acid encoding all or part of the rDNA gene and comprising a sequence of SEQ ID NO: 4. The amplifying means within the kit comprises a primer pair of oligonucleotides having the sequence of, for example, SEQ ID NO: 1 and SEQ ID NO: 6 or SEQ ID NO: 8 and SEQ ID NO: 11; and reagents necessary to perform PCR. The kit may also comprise a non-pathogen-specific oligonucleotide probe immobilized on solid matrix for amplifying a sequence having the sequence of SEQ ID NO: 5. A primer pair of oligonucleotides suitable for amplifying DNA from non-pathogenic Guignardia have the sequence of, for example, SEQ ID NO: 2 and SEQ ID NO: 6 respectively. Solid matrix supports suitable for such hybridizations and polymerizations are disclosed in U.S. Pat. No. 5,932,711, the entire disclosure of which is incorporated by reference herein. Preferably the primer pairs for amplifying pathogenic Guignardia are SEQ ID NOS: 8 and 11 and the primer pairs for amplifying non-pathogenic Guignardia are SEQ ID NOS: 9 and 10.

Various terms relating to the biological molecules and fungi of the present invention are used throughout the specification and claims.

With reference to nucleic acids used in the invention, the term “isolated nucleic acid” is sometimes employed. This term, when applied to DNA, refers to a DNA molecule that is separated from sequences with which it is immediately contiguous (in the 5′ and 3′ directions) in the naturally occurring genome of the organism from which it was derived. For example, the “isolated nucleic acid” may comprise a DNA molecule inserted into a vector, such as a plasmid or virus vector, or integrated into the genomic DNA of a procaryote or eucaryote. An “isolated nucleic acid molecule” may also comprise a cDNA molecule. An isolated nucleic acid molecule inserted into a vector is also sometimes referred to herein as a “recombinant” nucleic acid molecule.

With respect to RNA molecules, the term “isolated nucleic acid” primarily refers to an RNA molecule encoded by an isolated DNA molecule as defined above. Alternatively, the term may refer to an RNA molecule that has been sufficiently separated from RNA molecules with which it would be associated in its natural state (i.e., in cells or tissues), such that it exists in a “substantially pure” form (the term “substantially pure” is defined below).

The term “substantially pure” refers to a preparation comprising at least 50-60% by weight the compound of interest (e.g., nucleic acid, oligonucleotide, protein, etc.). More preferably, the preparation comprises at least 75% by weight, and most preferably 90-99% by weight, the compound of interest. Purity is measured by methods appropriate for the compound of interest (e.g. chromatographic methods, agarose or polyacrylamide gel electrophoresis, HPLC analysis, and the like).

With respect to antibodies, the term “immunologically specific” refers to antibodies that bind to one or more epitopes of a protein or compound of interest, but which do not substantially recognize and bind other molecules in a sample containing a mixed population of antigenic biological molecules.

With respect to single stranded nucleic acids, particularly oligonucleotides, the term “specifically hybridizing” refers to the association between two single-stranded nucleotide molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA or RNA molecule of the invention, to the substantial exclusion of hybridization of the oligonucleotide with single-stranded nucleic acids of non-complementary sequence. Appropriate conditions enabling specific hybridization of single stranded nucleic acid molecules of varying complementarity are well known in the art.

For instance, one common formula for calculating the stringency conditions required to achieve hybridization between nucleic acid molecules of a specified sequence homology is set forth below (Sambrook et al., 1989):

Tₘ=81.5° C.+16.6Log [Na+]+0.41(% G+C)−0.63(% formamide)−600/#bp in duplex

As an illustration of the above formula, using [Na+]=[0.368] and 50% formamide, with GC content of 42% and an average probe size of 200 bases, the Tm is 57° C. The Tₘ of a DNA duplex decreases by 1-1.5° C. with every 1% decrease in homology. Thus, targets with greater than about 75% sequence identity would be observed using a hybridization temperature of 42° C.

The term “oligonucleotide,” as used herein refers to primers and probes of the present invention, and is defined as a nucleic acid molecule comprised of two or more ribo- or deoxyribonucleotides, preferably more than three. The exact size of the oligonucleotide will depend on various factors and on the particular application and use of the oligonucleotide.

The phrase “consisting essentially of” when referring to a particular nucleotide acid means a sequence having the properties of a given SEQ ID No:. For example, when used in reference to a nucleic acid sequence, the phrase includes the sequence per se and molecular modifications that would not affect the basic and functional characteristics of the sequence. Accordingly, the probes and primers of the respective SEQ ID NOS: of the invention may be modified slightly, i.e., via the addition of about 5-25 nucleotides, or by the substitution of 1 to 5 nucleotides in the recited sequence. Despite such modifications, however, the sequences perform the function recited in the assay or kit.

The term “probe” as used herein refers to an oligonucleotide, polynucleotide or nucleic acid, either RNA or DNA, whether occurring naturally as in a purified restriction enzyme digest or produced synthetically, which is capable of annealing with or specifically hybridizing to a nucleic acid with sequences complementary to the probe. A probe may be either single-stranded or double-stranded. The exact length of the probe will depend upon many factors, including temperature, source of probe and use of the method. For example, for diagnostic applications, depending on the complexity of the target sequence, the oligonucleotide probe typically contains 15-25 or more nucleotides, although the number of nucleotides may vary. The probes herein are selected to be complementary to different strands of a particular target nucleic acid sequence. This means that the probes must be sufficiently complementary so as to be able to “specifically hybridize” or anneal with their respective target strands under a set of pre-determined conditions. Therefore, the probe sequence need not reflect the exact complementary sequence of the target. For example, a non-complementary nucleotide fragment may be attached to the 5′ or 3′ end of the probe, with the remainder of the probe sequence being complementary to the target strand. Alternatively, non-complementary bases or longer sequences can be interspersed into the probe, provided that the probe sequence has sufficient complementarity with the sequence of the target nucleic acid to anneal therewith specifically.

The phrase “solid matrix” as used herein includes, without limitation, filter paper, multiwell dishes, microchips, derivatized magnetic particles and the like.

The term “primer” as used herein refers to an oligonucleotide, either RNA or DNA, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as a suitable temperature and pH, the primer may be extended at its 3′ terminus by the addition of nucleotides by the action of a polymerase or similar activity to yield an primer extension product. The primer may vary in length depending on the particular conditions and requirement of the application. For example, in diagnostic applications, the oligonucleotide primer is typically 15-25 nucleotides in length, although this number may vary. The primer must be of sufficient complementarity to the desired template to prime the synthesis of the desired extension product, that is, to be able anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product.

Polymerase chain reaction (PCR) has been described in U.S. Pat. Nos. 4,683,195, 4,800,195, and 4,965,188, the entire disclosures of which are incorporated by reference herein.

### BRIEF DESCRIPTION OF THE DRAWINGS

FIG. 1 represents an rDNA sequence isolate associated with pathogenic Guignardia strains (SEQ ID NO: 4).

FIG. 2 an rDNA sequence isolate associated with non-pathogenic Citrus isolates (SEQ ID NO: 5).

### DETAILED DESCRIPTION OF THE INVENTION

Each of the cells of all life forms, except viruses, contain ribosomes and therefore ribosomal RNA. A ribosome contains four separate single-stranded RNA molecules, namely, a large molecule (26S), a medium sized molecule (18S), and two small molecules (5.8S and 5S). The two larger R-RNA molecules vary in size in different organisms.

Ribosomal RNA is a direct gene product and is coded for by the R-RNA gene. This DNA sequence is used as a template to synthesize R-RNA molecules. A separate gene exists for each of the ribosomal RNA subunits. Multiple R-RNA genes exist in most organisms and many higher organisms contain both nuclear and mitochondrial R-RNA genes. Plants and certain other organisms contain nuclear, mitochondrial and chloroplast R-RNA genes. The R-RNA gene and gene product have been well characterized in certain species. Hybridization of R-RNA and ribosomal genes in genetic analysis and evolution and taxonomic classification of organisms and ribosomal gene sequences has been described. Genetic analysis may include, for example, the determination of the numbers of ribosomal RNA genes in various organisms; the determination of the similarity between the multiple ribosomal RNA genes which are present in cells; determination of the rate and extent of synthesis of R-RNA in cells and the factors which control them.

In accordance with the present invention, molecular probes have been developed which distinguish between pathogenic and non-pathogenic Guignardia citricarpa species unambiguously, based on rDNA sequence differences. These probes may be used to advantage in methods routinely practiced in the laboratory setting.

Phylogenetically informative segments of ribosomal DNA from a large variety of Phyllosticta isolates have been sequenced in accordance with the present invention. The present invention provides a specific nucleic acid sequences isolated from Citrus fungus which differentiate Citrus fungal isolates which cause Black Spot symptoms from those which do not. The particular sequences studied to date include the internal transcribed spacer (ITS) 1 and ITS 2 segments of the Guignardia ribosomal DNA genes. From this work, it is clear that the pathogenic and non-pathogenic Guignardia citricarpa are quite different species with very different ITS sequences. Accordingly, species-specific PCR primers have been designed for use in an unequivocal and inexpensive method to differentiate between pathogenic and non-pathogenic Guignardia species.

I. Preparation of Nucleic Acid Molecules, Probes and Primers which Differentiate Between Pathogenic and Non-Pathogenic Guignardia Species

A. Nucleic Acid Molecules

Nucleic acid molecules encoding the differentiating oligonucleotides of the invention may be prepared by two general methods: (1) synthesis from appropriate nucleotide triphosphates, or (2) isolation from biological sources. Both methods utilize protocols well known in the art. The availability of nucleotide sequence information, such as a DNA having the sequence of SEQ ID NOS: disclosed herein enables preparation of an isolated nucleic acid molecule of the invention by oligonucleotide synthesis. Synthetic oligonucleotides may be prepared by the phosphoramidite method employed in the Applied Biosystems 38A DNA Synthesizer or similar devices. The resultant construct may be used directly or purified according to methods known in the art, such as high performance liquid chromatography (HPLC).

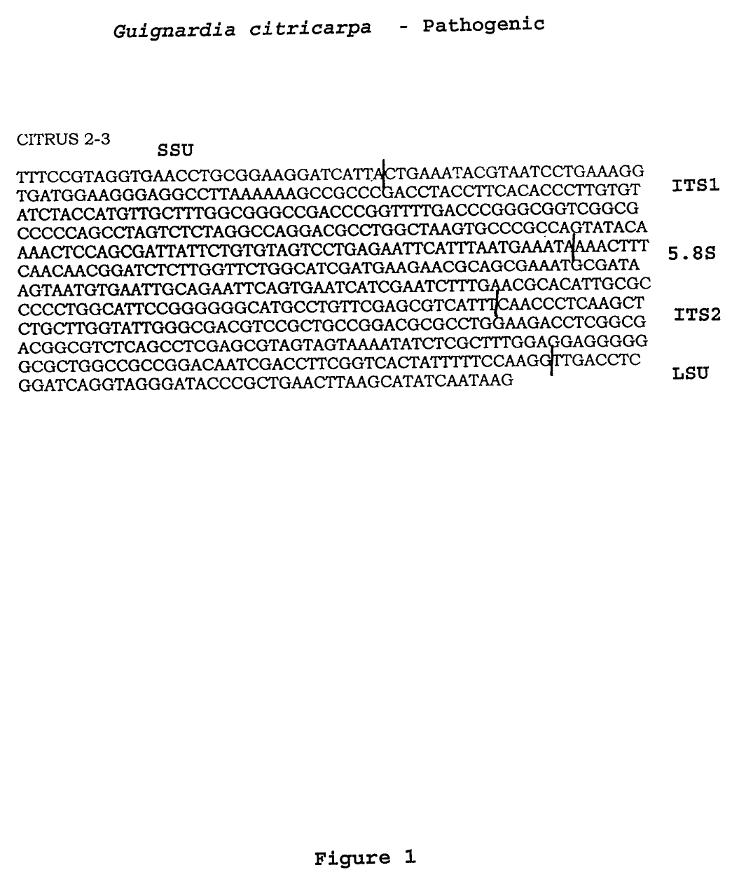

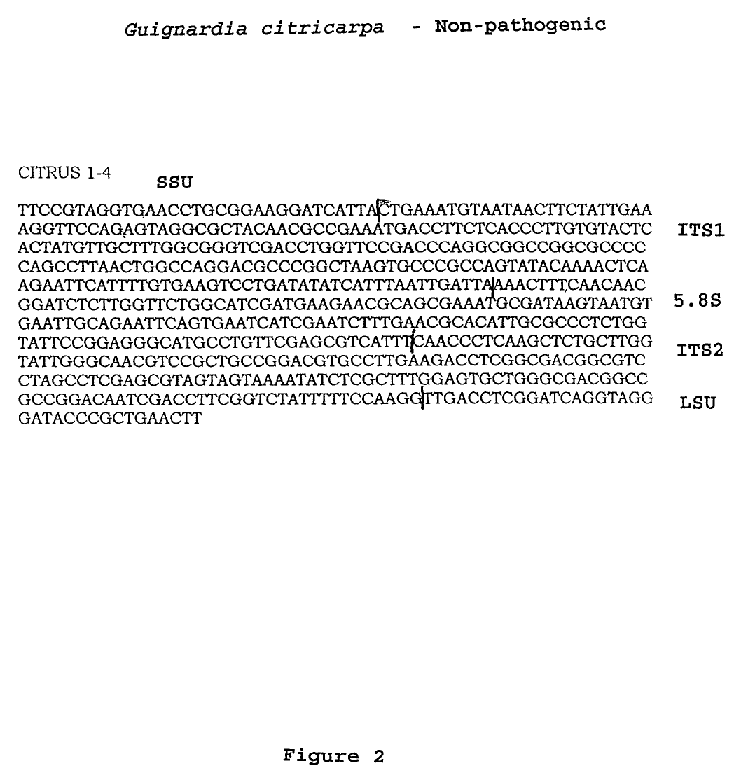

FIGS. 1 and 2 show the sequence differences between the ITS regions of pathogenic and non-pathogenic species of Guignardia. Accordingly, specific probes for identifying such sequences may be between 15 and 40 nucleotides in length. For probes longer than those described above, the additional contiguous nucleotides are provided within SEQ ID NOS: 4 and 5.

In accordance with the present invention, nucleic acids having the appropriate level of sequence homology with the sequences provided herein may be identified by using hybridization and washing conditions of appropriate stringency. For example, hybridizations may be performed, according to the method of Sambrook et al., Molecular Cloning, Cold Spring Harbor Laboratory (1989), using a hybridization solution comprising: 5×SSC, 5×Denhardt's reagent, 1.0% SDS, 100 μg/ml denatured, fragmented salmon sperm DNA, 0.05% sodium pyrophosphate and up to 50% formamide. Hybridization is carried out at 37-420° C. for at least six hours. Following hybridization, filters are washed as follows: (1) 5 minutes at room temperature in 2×SSC and 1% SDS; (2) 15 minutes at room temperature in 2×SSC and 0.1% SDS; (3) 30 minutes-1 hour at 370° C. in 1×SSC and 1% SDS; (4) 2 hours at 42-650° C. in 1×SSC and 1% SDS, changing the solution every 30 minutes.

The nucleic acid molecules of the invention include genomic DNA, RNA, and fragments thereof which may be single- or double-stranded. Thus, this invention provides oligonucleotides having sequences capable of hybridizing with at least one sequence of a nucleic acid molecule of the present invention, such as selected segments of the sequences provided herein. Also contemplated in the scope of the present invention are oligonucleotide probes which specifically hybridize with the DNA from pathogenic species of Guignardia while not hybridizing with DNA from the non-pathogenic species sequence under high stringency conditions. Primers capable of specifically amplifying the ITS segments of Guignardia rDNA encoding nucleic acids described herein are also contemplated to be within the scope of the present invention. As mentioned previously, such oligonucleotides are useful as primers for detecting, isolating and amplifying sequences associated with pathogenic Guignardia.

It will be appreciated by persons skilled in the art that variants (e.g., allelic variants) of the ITS sequences exist in the fungus population, and must be taken into account when designing and/or utilizing oligonucleotides of the invention. Accordingly, it is within the scope of the present invention to encompass such variants, with respect to the ITS sequences disclosed herein or the oligonucleotides targeted to specific locations on the respective genes or RNA transcripts. Accordingly, the term “natural allelic variants” is used herein to refer to various specific nucleotide sequences of the invention and variants thereof that would occur in a population. The occurrence of genetic polymorphisms which give rise to minor base changes in a DNA molecule are known to those of ordinary skill in the art. Additionally, the term “substantially complementary” refers to oligonucleotide sequences that may not be perfectly matched to a target sequence, but such mismatches do not materially affect the ability of the oligonucleotide to hybridize with its target sequence under the conditions described.

II. Detection OF Black Spot Disease-Associated Guignardia Sequences and Diagnostic Screening Assays Therefore

Currently, the most direct method for sequence analysis is DNA sequencing; however, it is also the most labor intensive and expensive method. Additionally, it is usually not practical to sequence all potentially relevant regions of every experimental sample. Other exemplary approaches for detecting the presence of pathogenic vs. non-pathogenic species of Guignardia based on nucleic acid differences include, for example:

a) comparing the sequence of nucleic acid in the sample with nucleic acid sequences from the non-pathogenic and pathogenic species of Guignardia to determine which species is present on the fruit; or

b) using DNA restriction mapping to compare the restriction pattern produced when a restriction enzyme cuts a sample of nucleic acid from the suspect fungus as compared with the restriction pattern obtained from pathogenic and non-pathogenic species of Guignardia; or

c) using a specific binding member capable of binding to either the non-pathogenic or pathogenic nucleic acid sequence, the specific binding member comprising nucleic acids which distinguish between the two species based on hybridization specificities, or alternatively comprising an antibody or antibody fragment with specificity for a pathogenic or non-pathogenic Guignardia nucleic acid sequence, the specific binding member being labeled so that binding of the specific binding member to its binding partner is detectable; or

d) in situ hybridization between lesion DNA from permeabilized tissue sections and the use of fluorescent molecular probes specific for pathogenic or non-pathogenic Guignardia species; or

e) using PCR involving one or more primers based on non-pathogenic or pathogenic Guignardia gene sequences to screen for the presence of the pathogenic species in a sample.

A “specific binding pair” comprises a specific binding member (sbm) and a binding partner (bp) which have a particular specificity for each other and which under normal conditions bind to each other in preference to other molecules. Examples of specific binding pairs are antigens and antibodies, ligands and receptors and complementary nucleotide sequences. The skilled person is aware of many other examples and do not need to be listed here. Further, the term “specific binding pair” is also applicable where either or both of the specific binding member and the binding partner comprise part of a large molecule. In embodiments in which the specific binding pair consists of nucleic acid sequences, such sequences will be of a length to hybridize to each other under conditions of the assay, preferably greater than 10 nucleotides long, more preferably greater than 15 or 20 nucleotides long.

In most embodiments for screening for the presence of pathogenic Guignardia, the nucleic acid associated with the pathogenic phenotype in the sample will initially be amplified, e.g. using PCR, to increase the amount of the analyte as compared to other sequences present in the sample. This allows the target sequences to be detected with a high degree of sensitivity if they are present in the sample. This initial step may be avoided by using highly sensitive array techniques that are becoming increasingly important in the art.

The identification of the nucleic acids associated with Guignardia pathogenicity paves the way for aspects of the present invention which provides the use of materials and methods, such as are disclosed and discussed above, for establishing the presence or absence in a test sample of the pathogenic species of the Guignardia fungus.

The invention also allows for planning of appropriate quarantine and/or prophylactic measures and permits stream-lining of analysis of infected Citrus fruits.

The following example is provided to illustrate an embodiment of the invention. It is not intended to limit the invention in any way. All temperatures are reported in centigrade degrees (° C.) unless otherwise noted.

### EXAMPLE I

### Isolation and Characterization of Pathogenic Guignardia ITS Sequences

The following experimental protocols are provided to facilitate the practice of the present invention.

Suspect Guignardia isolates were grown on 60 mm plastic petri plates containing a modified PDA medium: 20 g crude agar, 24 g Difco Potato Dextrose Broth, 1 g yeast extract, 1 g malt extract, 940 mL Distilled Water. After the medium had been autoclaved and cooled to approximately 65° C. the following liquid supplements were added before pouring the plates: 20 mL Antibiotic Stock to inhibit bacterial growth (5 mg/mL penicillin, 5 mg/mL streptomycin, 1.5 mg/mL chloramphenicol). Optionally, 10 mL Tricyclazole stock to inhibit melanin polymerization in cell walls (100 μg/mL) may be included. Additionally, 20 mL Ediphenphos stock to inhibit chitin formation in cell walls (2 mL/mL) may optionally be included. Cultures were optionally planted on inert membranes such as Nuclepore filters to facilitate removal of mycelium for extraction. Fungal isolates may alternatively be cultured in liquid culture, preferably in 5% carrot juice.

After three to five days of culture, 2-10 mg fresh weight of hyphae were scraped from the plate and placed into a 600 μL Eppendorf vial with 100 μL Epicentre Tfl (x1) buffer. The tissue was then subjected to 3 cycles of freezing (liquid nitrogen, 2 min) and heating (90°, 5 min). Next, 5 μL of extract were withdrawn from each sample and mixed with an equal volume of the following PCR reaction mix (total 100 μL) to amplify target rDNA: 4.0M Betaine (Acros Chemical Co.)-40 μL; Magnesium chloride, 25 mM -22 μL; dNTP's mixed, 2.5 mM, 16 μL; Tfl buffer (×20)-lOuL; Bovine serum albumin, 10 mg/mL, -5 μL; Distilled water -5.4 μL; Promega Taq polymerase, 1U/μL, 2 μL; reverse primer ITS 4(5′ TCCTCCGCTTATTGATATGC 3′; SEQ ID NO: 12), 10 MM, 0.8 μL; and forward primer ITS 5 (5′ GGAAGTAAAAGTCGTAACAAGG 3′; SEQ ID NO: 13), 10 mM, 0.8 μL. Other reagents are available for performing PCR including Epicentre MasterAmp which may be used in place of Betaine and Epicentre Tfl DNA polymerase which may be used in place of Promega Taq polymerase.

Each 10 μL reaction mix was loaded into a 10 μL capacity glass capillary tube; the ends were sealed and loaded into a RapidCycler (Idaho Technology) PCR machine and run with the following program: Hold: 94°, 15 sec. Cycle: Denature 94°, 1 sec; Anneal 55°, 3 sec; Elongation, 72°, 15 sec-slope 2.0, 39 cycles. Hold: 72°, 1 minute. The contents of the capillary tube were loaded into a PCR vial, and a second PCR reaction was prepared to amplify the pathogenic Guignardia sequence (if present) as follows. Mix A: 52 μL distilled water; 10 μL Tfl buffer (×20); 2 μμL Epicentre Tfl DNA polymerase. Mix B. 40 μL Tfl MasterAmp (×10); 22 μL magnesium chloride; 17 μL distilled water; 16 μL dNTP's; 5 μL BSA. To a 200 μL PCR tube add 5 μL Mix B, 3.2 μL Mix A, 0.8 μL CIT 1 ITS forward primer (5′ AAAAAGCCGCCCGACCTACCT 3′; SEQ ID NO: 1), 0.8 μL reverse primer ITS 2 (5′ GCTGCGTTCTTCATCGATGC 3′ (SEQ ID NO: 6), and 0.2-0.3 μL template DNA taken directly from the previous PCR reaction mix. The sample is then run on a Rapid Cycler using the same program as the initial PCR reaction. Alternatively, SEQ ID NOS: 8 and 6 may be used as the forward and reverse primers respectively.

Following completion of the thermocycling program, a 2% agarose gel (electrophoresis grade) prepared with a narrow comb was loaded with 3-5 μL of sample in 4-5 μL of loading buffer (total volume 7-10 μL) and run in TBE buffer at 80 V for 30 minutes. Following electrophoresis the entire gel was stained with SYBAR Gold (Molecular Probes), 1:10,000 dilution for 15 minutes. The gel was placed on a Clare Dark Reader. A positive reaction was evident as a bright band on the order of 150 bp. If the band was present, it was concluded that the Guignardia species under investigation is pathogenic. To ensure this observation, the primers specific for the non-pathogenic species were substituted for CIT1-ITS1. These primer pairs specifically amplify DNA from the non-pathogenic Guignardia species and have the following sequences. CIT3-ITS1 forward primer (5′ GCTACAACGCCGAAATGACCTT 3′; SEQ ID NO: 2) and ITS2 reverse primer (5′ GCTGCGTTCTTCATCGATGC 3′; SEQ ID NO: 6) or ITS3 forward primer (5′ GCATCGATGAAGAACGCAGC 3′; SEQ ID NO: 7) and CIT3-ITS2R reverse primer (5′ GCCGTCGCCCAGCACTC 3′; SEQ ID NO: 3). FIG. 1 shows the rDNA ITS sequence associated with pathogenic Guignardia , SEQ ID NO: 4. FIG. 2 shows the rDNA ITS sequence associated with non-pathogenic Guignardia , SEQ ID NO: 5.

### EXAMPLE II

### Alternative Isolation of Guignardia DNA

A sample of Guignardia from a Citrus fruit was cultured by inoculation of the sample into a solution of 5% aqueous carrot juice. Following a suitable incubation period (typically between 72 hours and 1 week at 25° C.), mycelia were removed from the culture medium and rinsed with distilled water. The rinsed mycelia were added to PCR buffer and heated at 90° C. for 10 minutes to release fungal DNA. An aliquot (1/10 of the total volume) of the heated fungal DNA-containing PCR buffer was amplified as described in Example I. The amplified sample was separated by gel electrophoresis, stained, and read according to the methods described in Example I.

A further refinement of this method may be achieved utilizing sequence specific fluorescent probes which bind to the middle of the sequence during PCR. These are known as “Molecular Beacons”, and would confer greater sensitivity and specificity on the procedure described above. Additionally, the fluorescence could be directly detected in the PCR mix thereby obviating the need for detecting bands on agarose gels.

### REFERENCES

1. Carroll, G. C., (1992) Fungal Mutualism. In The Fungal Community. Eds. G. C. Carroll and D. T. Wicklow pages 327-354. New York; Marcel Denker, Inc.

2. Kotzé, J. D. (1981) Epidemiology of Control of Citrus Black Spot in South Africa. Plant Disease 65: 12.

3. McOnie, K. C. (1964) The Latent Occurrence on Citrus and other hosts of Guignardia. Phytopathology 54:40-43.

4. H. A. van der Aa (1973) Studies on Phyllosticta I. Studies in Mycology #5, CBS, Baarn, The Netherlands.

Sequences Utilized in the Present Invention

SEQ ID NOS: 1 and 8—Forward primers for amplification of pathogenic Guignardia species

SEQ ID NOS: 2, 3, 7 and 9—Forward primers for amplification of non-pathogenic Guignardia species.

SEQ ID NO: 4—rRNA sequence from pathogenic Guignardia species

SEQ ID NO: 5—rRNA sequence from non-pathogenic Guignardia species

SEQ ID NOS: 6, 10 and 11—Reverse primers for amplification of both pathogenic and non-pathogenic Guignardia species

SEQ ID NOS: 12 and 13—Forward and reverse primers for the amplification of internal transcribed spacer regions from Guignardia.

While a preferred embodiment of the present invention has been described and specifically exemplified above, it is not intended that the invention be limited to such an embodiment. Various modifications may be made thereto without departing from the scope and spirit of the present invention, as set forth in the following claims.

What is claimed is:

1. An oligonucleotide which selectively hybridizes with a target DNA sequence associated with pathogenic species of Guignardia, said oligonucleotide having a sequence selected from the group consisting of

| | |
|---|---|
| AAAAAGCCGCCCGACCTACCT | (SEQ ID NO: 1) and |
| TAAAAAAGCCGCCCGACCTAC | (SEQ ID NO: 8). |

2. An oligonucleotide which selectively hybridizes with non-pathogenic species of Guignardia, said oligonucleotide being selected from the group consisting of GCTACAACGCCGAAATGACCTT (SEQ ID NO: 2),

| | |
|---|---|
| GCCGTCGCCCAGCACTC | (SEQ ID NO: 3), and |
| GCTACAACGCCGAAATGACC | (SEQ ID NO: 9). |

3. An oligonucleotide for priming a DNA amplification of a target DNA sequence associated with pathogenic species of Guignardia, said oligonucleotide having a sequence selected from the group consisting of

| | |
|---|---|
| AAAAAGCCGCCCGACCTACCT | (SEQ ID NO: 1) and |
| TAAAAAAGCCGCCCGACCTAC | (SEQ ID NO: 8). |

4. An oligonucleotide for priming a DNA amplification of a target DNA sequence associated with non-pathogenic species of Guignardia, said oligonucleotide being selected from the group consisting of GCTACAACGCCGAAATGACCTT (SEQ ID NO: 2),

| | |
|---|---|
| GCCGTCGCCCAGCACTC | (SEQ ID NO: 3), and |
| GCTACAACGCCGAAATGACC | (SEQ ID NO: 9). |

5. A method for priming DNA amplification of a target DNA sequence associated with pathogenic species of Guignardia, said method comprising:
a) obtaining a DNA sample from a fruit suspected of being infected with Guignardia;
b) providing a forward primer selected from the group consisting of SEQ ID NO: 1 and 8;
c) providing a reverse primer selected from the group consisting of SEQ ID NO: 6 and 11; and
d) subjecting said DNA sample and said forward and reverse primers to conditions suitable for polymerase chain reaction, amplification of said DNA in the presence of said primers indicating infection with a pathogenic Guignardia species.

6. A method for differentiating pathogenic species of Guignardia from non-pathogenic species of Guignardia, comprising the steps of:
a) obtaining a DNA sample from a Citrus fruit suspected of being infected with Guignardia;
b) contacting said DNA with a detectably labeled probe which selectively hybridizes with said DNA, said probe having the sequence of SEQ ID NO: said probe having a sequence selected from the group consisting of SEQ ID NO: 1, SEQ ID NO: 2, SEQ ID NO: 3, SEQ ID NO: 8, AND SEQ ID NO:9; and detecting specific hybridization if any, samples demonstrating hybridization with SEQ ID NO: 1 or SEQ ID NO: 8 being indicative infection of the Citrus fruit with pathogenic Guignardia and samples demonstrating hybridization with SEQ ID NOS: 2, 3, or 9 being indicative of infection of the Citrus fruit with non-pathogenic species of Guignardia.

7. A method for differentiating pathogenic species of Guignardia from non-pathogenic species of Guignardia, comprising the steps of:
a) obtaining a DNA sample from a Citrus fruit suspected of being infected with Guignardia;
b) immobilizing said DNA sample on a solid support;
c) probing said immobilized sample DNA with a detectably labeled probe, said probe having a sequence selected from the group consisting of SEQ ID NO: 1, SEQ ID NO: 2, SEQ ID NO: 3, SEQ ID NO: 8, AND SEQ ID NO:9; and
d) assessing said solid support for hybridization of said probe to said immobilized DNA, samples demonstrating hybridization with SEQ ID NO: 1 or SEQ ID NO: 8 being indicative infection of the Citrus fruit with pathogenic Guignardia and samples demonstrating hybridization with SEQ ID NOS: 2, 3, or 9 being indicative of infection of the Citrus fruit with non-pathogenic species of Guignardia.

8. A method of screening for Citrus Black Spot disease, said method comprising
a) obtaining a nucleic acid sample; and
b) assaying the nucleic acid sample for the presence of a sequence which hybridizes with SEQ ID NO: 1 or SEQ ID NO: 8 or SEQ ID NO: 4, wherein the presence of said hybridizing sequence is indicative of pathogenic Guignardia infection causing Citrus Black Spot disease.

9. A method in accordance with claim 5, wherein said assaying further comprises amplification of the ITS region of Guignardia rDNA containing an ITS nucleic acid, said ITS nucleic acid sequence being amplifiable using primers consisting of SEQ ID NO: 12 and SEQ ID NO: 13.

10. A method in accordance with claim 5, wherein said polymerase chain reaction is primed by SEQ ID NOS: 8 and 11.

11. A kit for use in screening for Citrus Black Spot disease comprising a pathogen-specific oligonucleotide probe immobilized on solid matrix, and further comprising means for amplifying a test samples's nucleic acid encoding all or part of the rDNA gene, wherein said test sample's nucleic acid comprises a sequence of SEQ ID NO: 4.

12. A kit in accordance with claim 11, wherein said amplifying means comprises:
(a) a primer pair of oligonucleotides comprising a first oligonucleotide having the sequence of SEQ ID NO: 1 or SEQ ID NO: 8 and a second oligonucleotide having the sequence of SEQ ID NO: 6 or SEQ ID NO: 11; and
(b) reagents necessary to perform PCR.

13. A kit in accordance with claim 12, wherein the oligonucleotide pair comprises SEQ ID NO: 8 and SEQ ID NO: 11.

14. A kit for use in screening for non-pathogenic Guignardia species comprising a non-pathogen-specific oligonucleotide probe immobilized on solid matrix, and further comprising means for amplifying a test samples's nucleic acid encoding all or part of the rDNA gene, wherein said nucleic acid comprises a sequence of SEQ ID NO: 5.

15. A kit in accordance with claim 14, wherein said amplifying means comprises:
(a) a primer pair of oligonucleotides comprising a first oligonucleotide having the sequence of SEQ ID NO: 2 or SEQ ID NO: 9 and a second oligonucleotide having the sequence of SEQ ID NO: 6 or SEQ ID NO:10 or SEQ ID NO: 11; and
(b) reagents necessary to perform PCR.

16. A kit in accordance with claim 15 wherein the oligonucleotide pair comprises SEQ ID NO: 9 and SEQ ID NO: 10.

17. A method for identifying pathogenic Guignardia species in a sample, said method comprising the steps of:
a) culturing G. citricarpa;
b) subjecting said cultured G. citricarpa to conditions which effect lysing of said G. citricarpa, thereby releasing DNA from said hyphae;
c) contacting said released DNA with a primer pair of oligonucleotides comprising a first oligonucleotide having the sequence of SEQ ID NO: 1 or SEQ ID NO: 8 and a second oligonucleotide having the sequence of SEQ ID NO: 6 or SEQ ID NO: 11 under conditions where amplification of pathogenicity-associated ITS sequences occurs, if said pathogenic Guignardia is present in said sample; and
d) detecting said amplified sequence, if present.

18. A method as claimed in claim 17, wherein said amplified sequence is detected via incorporation of a detectable label.

19. A method as claimed in claim 17, wherein said amplified sequence is detected by gel electrophoresis of said amplified sample.

20. A method as claimed in claim 17, wherein said ITS sequences are amplified using a primer set having SEQ ID NOS: 12 and 13 prior to amplification of pathogenicity related sequences employing a primer set having SEQ ID NO: 8 and 11.

21. A method for identification of pathogenic Guignardia citricarpa, said method comprising:
a) contacting a tissue section containing a black spot lesion with a permeabilization agent;
b) contacting said permeabilized lesion DNA with a detectably labeled oligonucleotide having a sequence of SEQ ID NO: 1 or SEQ ID NO: 8; and
c) detecting hybridization of said oligonucleotide to said DNA, if any, said hybridization indicating the presence of pathogenic G. citricarpa.
